(19) United States
(12) Patent Application Publication
(10) Pub. No.: US 2026/0137475 A1
(43) Pub. Date: May 21, 2026

(54) METHOD OF MANUFACTURING A CONTAINER, SYSTEM AND METHOD FOR HANDLING OF A SURGICAL INSTRUMENT

(71) Applicant: OLYMPUS Winter & Ibe GmbH, Hamburg (DE)

(72) Inventors: Dennis AEHLIG, Hamburg (DE); Stefan SCHROEDER, Hamburg (DE)

(73) Assignee: OLYMPUS Winter & Ibe GmbH, Hamburg (DE)

(21) Appl. No.: 19/446,142

(22) Filed: Jan. 12, 2026

Related U.S. Application Data
(62) Division of application No. 18/225,820, filed on Jul. 25, 2023.
(60) Provisional application No. 63/397,443, filed on Aug. 12, 2022.

(51) Int. Cl. A61B 50/13; A61B 50/10; A61B 50/18; A61B 50/30; A61B 50/39; A61L 31/08; A61L 31/16

(52) U.S. Cl. CPC A61B 50/13; A61B 50/30; A61L 31/088; A61L 31/16; A61B 2050/105; A61B 2050/185; A61B 50/39; A61L 2300/404

(57) ABSTRACT

A method for handling of a surgical instrument in a healthcare facility during a reprocessing cycle. The method including: reprocessing the surgical instrument in at least one reprocessing apparatus for cleaning the surgical instrument, transporting the cleaned surgical instrument from the at least one reprocessing apparatus to a storage cabinet with a transport container comprising at least one first wall having a first inner surface defining a first storage space for accommodating the surgical instrument, and storing the transported surgical instrument in a storage cabinet comprising at least one second wall having a second inner surface defining a second storage space for accommodating the surgical instrument. Wherein at least a portion of the first inner surface and second inner surface of the storage cabinet and the transport container is an antimicrobial surface.

### CROSS-REFERENCE TO RELATED APPLICATIONS

The present application is a divisional application of U.S. patent application Ser. No. 18/225,820 filed on Jul. 25, 2023, which is based upon and claims the benefit of priority from U.S. Provisional Application No. 63/397,443 filed on Aug. 12, 2022, the entire contents of each of which is incorporated herein by reference.

### BACKGROUND

### Field

The present disclosure relates to method of manufacturing a container for storage and/or transport of a disinfected surgical instrument and a method for handling of a surgical instrument in a healthcare facility during a reprocessing cycle.

### Prior Art

Surgical instruments, for example flexible endoscopes, have to be reprocessed after being used in a surgery to prevent infection of patients. The most common method to reprocess endoscopes is to submit them to a high-level disinfection process. After the disinfection process, the endoscopes need to be dried and stored. To do this, they are usually transported to a storage area. In the storage area, the endoscopes are placed in a storage cabinet for drying and storage, where they may stay for several weeks until they are required in the next surgery.

However, there remains the risk of recontamination of the surgical instruments after the reprocessing process. For example, dirt and microorganisms may come into contact with a surface of the surgical instrument during transport or storage. The use of such a recontaminated surgical instrument in a surgery poses a serious health risk to a patient.

In order to avoid recontamination, the transport containers used to transport the endoscopes and the storage cabinets are usually cleaned and disinfected manually in regular intervals. The chemicals utilized for this are often quite aggressive to ensure that the storage cabinets and transport containers are disinfected properly. However, the use of such chemicals poses a health risk to the medical personal and may damage the storage cabinets and transport containers themselves. Also, the task of cleaning and disinfecting is time-consuming and prone to mistakes, as a single spot not disinfected correctly may be enough to recontaminate the surgical instrument.

### SUMMARY

An object of the present disclosure is to prevent a recontamination of surgical instruments after a reprocessing process.

Such object can be solved by a container configured for storage and/or transport of a disinfected surgical instrument, comprising a storage space for accommodating the surgical instrument, the storage space being confined by at least one wall having an inner surface facing the storage space, wherein at least a section of the inner surface is an antimicrobial surface.

The at least a section of the inner surface as an antimicrobial surface prevents a contamination of any surgical instrument coming into contact with this section. The antimicrobial coating substitutes a disinfection of a respective surface. Hence, a manual disinfection of the inner surface is no longer necessary. The at least one wall of the container may be a side wall and/or a bottom wall and/or a lid and/or a top and/or a door of the container. The walls can most likely come into contact with the surgical instrument can be configured to have an inner surface that is the antimicrobial surface. For example, if the surgical instrument is usually placed on the bottom of the storage space, the inner surface of the bottom wall can be the antimicrobial surface. If there is a risk that the surgical instrument may also come into contact with the side walls of the storage space, the inner surfaces of the side walls can also be antimicrobial surfaces. If the surgical instrument may only come into contact with a section of the bottom wall, only this section can be an antimicrobial surface.

The antimicrobial surface can be a hydrophilic surface or a hydrophobic surface. A hydrophobic surface can prevent the adherence of dirt. In this way, a recontamination for example, by dirt on the inner surface can be avoided. A hydrophilic surface does not prevent the adherence of dirt directly. However, such a surface can still be useful in hygienic applications as the cleaning can be facilitated by the hydrophilic character of the surfaces. The hydrophilic effect can be used when washing or spraying is applied as a cleaning method. The water and/or a cleaning agent, which is applied to a hydrophilic surface, can better wet or attach to it and thereby washing and cleaning away dirt can be easier.

In case of an antimicrobial surface, which should be a “disinfection-free” surface, a hydrophobic coating can be used.

The antimicrobial surface can be a plasma activated surface. For example, by treating the inner surface with plasma activation, a hydrophilic surface or a hydrophobic surface can be achieved. In addition, plasma activation can clean the inner surface from organic contamination and prevent adherence of biomatter. Furthermore, plasma activation can be a fast, cost-effective and reliable method to create an antimicrobial surface.

The antimicrobial surface can comprise an antimicrobial coating, wherein the antimicrobial coating can comprise copper and/or silver, wherein the antimicrobial coating can be applied by chemical vapor deposition and/or physical vapor deposition.

The antimicrobial coating can provide antimicrobial properties to most materials and effectively prevent a recontamination. A coating that incorporates copper and/or silver can act efficiently against microorganisms. Chemical vapor deposition and physical vapor deposition can be used for creation of the antimicrobial coating, as they can coat large areas and can be applied to heat-sensitive materials, for example plastics. The antimicrobial coating can comprise a matrix material, wherein a bioactive agent, for example copper and/or silver, can be embedded in this matrix. The matrix material and/or the bioactive agent, i.e., copper and/or silver, can be deposited by one and the same or via different deposition technologies.

The antimicrobial coating can be optically transparent. The antimicrobial surface can be translucent. By applying an optically transparent and/or translucent antimicrobial coating on the inner surface, the visual properties of the container are not affected. This allows, for example, to differentiate two containers by color, despite both containers having identical coatings.

According to an embodiment, the container can be a transport container and the antimicrobial surface can be a surface of a bottom wall and/or at least one side wall and/or a top wall of the storage space of the transport container.

Such transport containers can be utilized to transport the disinfected surgical instrument from a surgical area to the reprocessing area, from the reprocessing area to a storage area and from the storage area back to the surgical area. By providing the inner surface of these transport containers with antimicrobial properties, a contamination of the inner surface can be prevented. Otherwise, the inner surface might become contaminated while transporting a used surgical instrument to the reprocessing area. If the same transport container is used afterwards to transport a disinfected surgical instrument, the disinfected surgical instrument may come into contact with the same wall. By configuring at least a section of the inner wall as an antimicrobial surface, a recontamination of the disinfected surgical instrument can be prevented even if the disinfected surgical instrument comes into contact with the wall, which previously came into contact with the used surgical instrument.

The transport container can be, for example, a tray, which can be placed in a transport wagon. To prevent recontamination, the inside of the tray can be an antimicrobial surface. The transport wagon itself can also be a transport container. For example, a top wall of the storage area of the transport wagon, in which the tray is placed, can comprise an inner surface that is an antimicrobial surface. In this way, contamination of the surgical instrument can be prevented when it accidently hits the top wall of the transport wagon during transport.

According to another embodiment, the container can be a storage cabinet and the antimicrobial surface can be a surface of a bottom wall and/or at least one side wall and/or a top wall and/or a door of the storage space.

A storage cabinet can be used for storage and for drying of the disinfected surgical instrument. Recontamination during storage can be prevented by the antimicrobial surface in the storage cabinet. Especially when introducing new surgical instruments into the storage space or taking out surgical instruments from the storage space, some of the surgical instruments may come into contact with the walls of the storage space. However, the antimicrobial surface can prevent a recontamination when this happens. Also, when accidently putting a contaminated surgical instrument into the storage space, an adherence of dirt and microorganism on the walls of the storage space does not occur.

The container can be configured to transport flexible endoscopes and/or store flexible endoscopes during a drying process. The container can be used both for transport and for storage. Thus, the container may be both a transport container and storage cabinet.

Such object can be further solved by a method of manufacturing a container for storage and/or transport of a disinfected surgical instrument, the container comprising a storage space for accommodating the surgical instrument, the storage space being confined by at least one wall having an inner surface facing the storage space, wherein an antimicrobial surface treatment is applied to at least a section of the inner surface.

The same or similar advantages apply to the method of manufacturing a container as were previously mentioned with respect to the container.

The antimicrobial surface treatment can be a hydrophilic surface treatment or a hydrophobic surface treatment and/or a plasma activation treatment.

An antimicrobial coating can be applied to the section of the inner surface, such as by chemical vapor deposition and/or physical vapor deposition, wherein the coating can comprise copper and/or silver.

According to an embodiment, the antimicrobial coating can be applied on a semi-finished part of the container prior to final assembly to facilitate assembly of the container.

Such object can be further solved by a system for handling of a surgical instrument in a healthcare facility during a reprocessing cycle, the system comprising a plurality of care cycle apparatuses each configured to accommodate the surgical instrument during the reprocessing cycle, the plurality of care cycle apparatuses comprising at least one first care cycle apparatus and at least one second care cycle apparatus, wherein the first care cycle apparatus is a storage cabinet and the second care cycle apparatus is a transport container, wherein the care cycle apparatuses each comprise a storage space for accommodating the surgical instrument, wherein at least one wall confining the storage space of every care cycle apparatus comprises an inner surface that is an antimicrobial surface.

The same or similar advantages apply to the system for handling of a surgical instrument in a healthcare facility as were previously mentioned with respect to the method of manufacturing a container and the container.

A reprocessing cycle includes the necessary steps to reprocess a used surgical instrument and making it available for another surgery. The reprocessing cycle may also be called a care cycle. During the reprocessing cycle, after the surgical instrument is cleaned and disinfected, a recontamination needs to be prevented before it is used again in surgery. As it is necessary to dry and store the surgical instrument as well as transport it to different areas in the healthcare facility, any risks of recontamination during these steps have to be eliminated/minimized. For this reason, all the care cycle apparatuses used to accommodate the surgical instrument after reprocessing can have an antimicrobial surface on the inner surfaces of the walls confining the storage space. These care cycle apparatuses are, for example, transport containers and storage cabinets or any other containers used to accommodate the surgical instrument during the reprocessing cycle after disinfection and cleaning.

The storage space of each care cycle apparatus can be configured to accommodate the surgical instrument in that the surgical instrument may only come into contact with antimicrobial surfaces of the storage spaces during the reprocessing cycle. The plurality of care cycle apparatuses can comprise at least one reprocessing apparatus.

Such object can be further solved by a method for handling of a surgical instrument in a healthcare facility during a reprocessing cycle, wherein the surgical instrument is accommodated in a plurality of care cycle apparatuses at different times during the reprocessing cycle, wherein the plurality of care cycle apparatuses comprise at least one first care cycle apparatus and at least one second care cycle apparatus, wherein the first care cycle apparatus is a storage cabinet and the second care cycle apparatus is a transport container, wherein the care cycle apparatuses each comprise a storage space for accommodating the surgical instrument, wherein the method comprises:
reprocessing the surgical instrument in at least one reprocessing apparatus,
transporting the surgical instrument from the reprocessing apparatus to the storage cabinet with the transport container, and
storing the surgical instrument in the storage cabinet after reprocessing the surgical instrument,
wherein at least one wall confining the storage space of every care cycle apparatus comprises an inner surface that is an antimicrobial surface.

The same or similar advantages apply to the method for handling of a surgical instrument as were previously mentioned with respect to the system for handling of surgical instruments, the method of manufacturing a container and the container.

The method can eliminate any risk of recontamination of the reprocessed surgical instrument by the inner surfaces of the storage spaces of the care cycle apparatuses.

The method can further comprise one or more of the following:
prior to reprocessing the surgical instrument, transporting the surgical instrument to the reprocessing apparatus with the transport container,
after reprocessing the surgical instrument, drying the surgical instrument in the storage cabinet,
transporting the surgical instrument with the transport container from the storage cabinet to a surgical area for use in a surgery.

### BRIEF DESCRIPTION OF THE DRAWINGS

Further characteristics will become apparent from the description of the embodiments together with the claims and the included drawings. Embodiments can fulfill individual characteristics or a combination of several characteristics.

The embodiments are described below, without restricting the general intent of the invention, based on exemplary embodiments, wherein reference is made expressly to the drawings with regard to the disclosure of all details that are not explained in greater detail in the text. In the drawings:

FIG. 1 illustrates a schematic simplified representation of a system for handling of a surgical instrument in a healthcare facility during a reprocessing cycle,

FIG. 2 illustrates a schematic perspective representation of a container for a surgical instrument with an antimicrobial inner surface,

FIG. 3 illustrates a schematic perspective representation of a container with a partial antimicrobial inner surface,

FIG. 4 illustrates a schematic perspective representation of a storage cabinet with an antimicrobial surface on its bottom wall, its side walls and its doors, and

FIG. 5 illustrates a schematic simplified representation of a reprocessing cycle of a surgical instrument.

In the drawings, the same or similar types of elements or respectively corresponding parts are provided with the same reference numbers in order to prevent the item from needing to be reintroduced.

### DETAILED DESCRIPTION

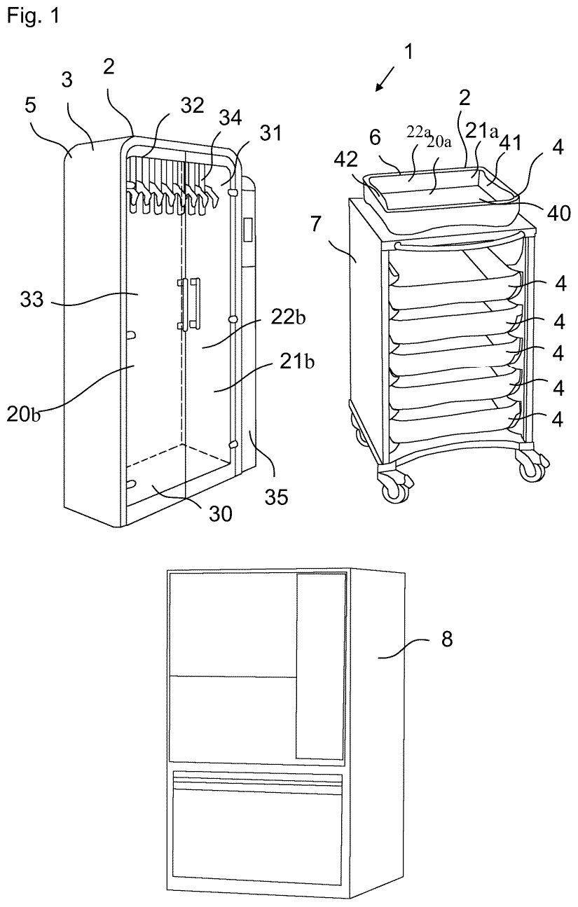

FIG. 1 shows a schematic representation of an exemplary embodiment of a system 1 for handling of a surgical instrument 10 in a healthcare facility during a reprocessing cycle. The surgical instrument 10 is not shown in FIG. 1. The system 1 comprises a plurality of care cycle apparatuses 5, 6 and a reprocessing apparatus 8. The first care cycle apparatus 5 is a storage cabinet 3 and the second care cycle apparatus 6 is a transport container 4. Both the storage cabinet 3 and the transport container 4 are containers 2 for accommodating surgical instruments 10.

After use in surgery, the surgical instrument 10 is cleaned and/or disinfected in the reprocessing apparatus 8. Afterwards it is transported with the transport container 4, which in this example is a tray, to the storage cabinet 3 for drying and storage. During transport, the surgical instrument 10 is placed in a storage space 20a of the transport container 4. The transport container 4 has a bottom wall 40, four side walls 41 and a top wall 42 at its upper edge. In the exemplary embodiment, the bottom wall 40 and the side walls 41 are the walls 21a confining the storage space 20a. During transport, the surgical instrument 10 may come into contact with an inner surface 22a of these walls 21a. If these inner surfaces 22a are contaminated by dirt or microorganisms, the surgical instrument may become recontaminated, which poses a health risk for the patient.

After receiving the surgical instrument 10, the transport container 4 is placed in a transport wagon 7, which can hold several transport containers 4. The transport wagon 7 has for example four wheels, so that the medical personal can move the stored transport containers 4 to the areas where they are needed.

For drying and storage of the surgical instrument 10, it is placed in the storage space 20b of the storage cabinet 3. The storage space 20b of the storage cabinet 3 comprises a bottom wall 30, three side walls 31, a top wall 32 and doors 33, which form the walls 21b of the storage space 20b. Inside the storage space 20b, a number of holders 34 are provided, which can hold the surgical instruments 10, for example flexible endoscopes. A drying attachment 35 helps with the drying of the surgical instruments 10 in the storage space 20b.

However, when a surgical instrument 10 is placed into the storage space 20b or removed from it, the surgical instrument 10 may come into contact with an inner surface 22b of the walls 21b. If there are dirt or microorganisms on these walls 21b, the surgical instrument 10 may become recontaminated during this contact.

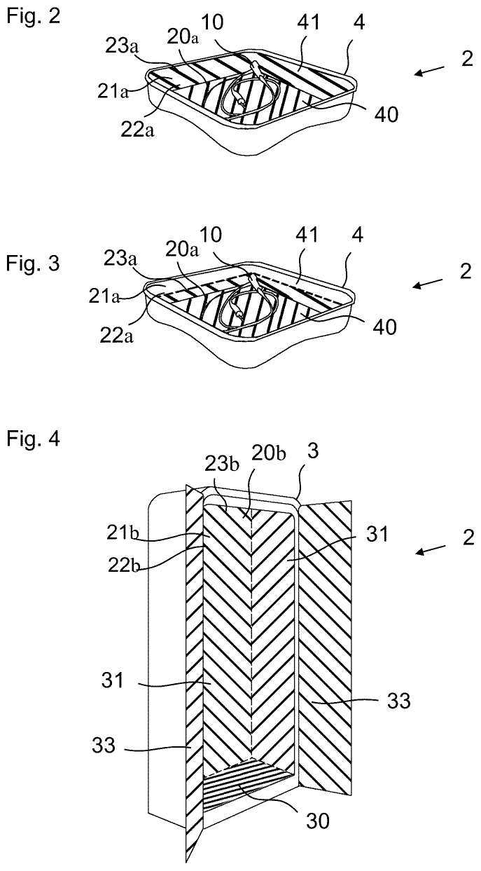

FIG. 2 shows schematically a container 2, which in this exemplary embodiment is one of the transport containers 4 formed as a tray. A surgical instrument 10 is accommodated in the storage space 20a of the transport container 4. To prevent recontamination during transport of the surgical instrument 10, the inner surface 22a of the walls 21a are provided with an antimicrobial surface 23a, which is represented as dashed lines. In order to create the antimicrobial surface 23a, the inner surface 22a may be treated with plasma activation and coated with an antimicrobial coating, for example copper and/or silver. The plasma activation prevents dirt from adhering to the inner surface 21a and the antimicrobial coating destroys microorganisms. In addition, the transport wagon 7 may be provided with an antimicrobial surface 23a as well, for example on any parts which may come into contact with the surgical instrument 10 during transport of the transport container 4 inside the transport wagon 7. As such, the transport wagon 7 may be considered to be a transport container 4 as well.

FIG. 3 shows another exemplary embodiment of a transport container 4 with an antimicrobial surface 23a. In this example, the walls 21a confining the storage space 20a are only partially covered by the antimicrobial coating shown in dashed lines. By configuring only a section of the inner surface 22a as an antimicrobial surface 23a, the costs of manufacturing the transport container 4 are reduced. To still protect the surgical instrument 10 from recontamination, the antimicrobial surface 23a is only omitted from sections of the inner surface 22a, which do not come into contact with the surgical instrument 10 during transport.

FIG. 4 shows schematically a storage cabinet 3 as another example of a container 2 with an antimicrobial surface 23b. Unlike in FIG. 1, the doors 33 of this storage cabinet 3 are open and not transparent. Also, the holders 34 are not shown in FIG. 4 for better visibility. As can be seen in FIG. 4, all the walls 21b of the storage space 20b are provided with an antimicrobial surface 23b on their inner surface 22b. This includes the inner surfaces 22b of the bottom wall 30, the three side walls 31 and the insides of the doors 33, as represented by the dashed lines. The top wall 32, which is not visible in FIG. 4, may also be provided with an antimicrobial surface 23b. If the storage cabinet 3 also includes holders 34, the surface of the holders 34 may also be provided with an antimicrobial surface 23b.

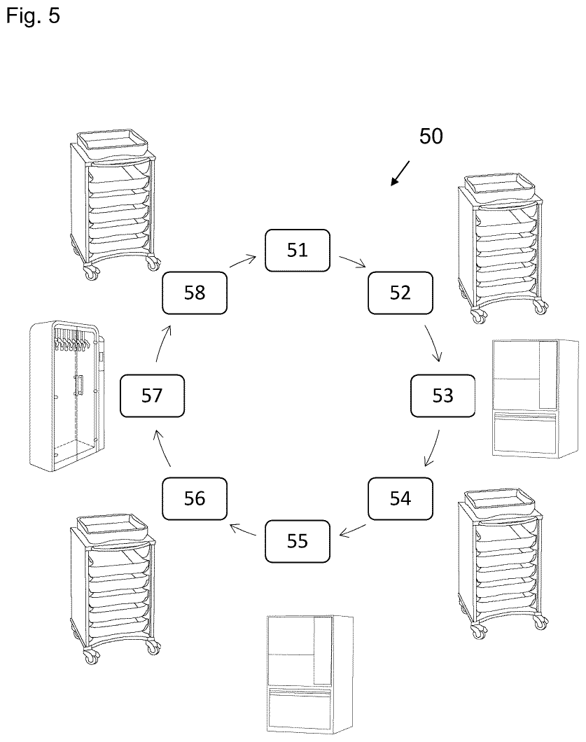

FIG. 5 shows schematically and in a simplified manner an exemplary reprocessing cycle 50 for a surgical instrument 10. At the surgery step 51, the surgical instrument 10 is used at a surgery area for a surgery, during which it becomes contaminated. Afterwards, it is transported from the surgery area to a reprocessing area during a first transport step 52. During a first reprocessing step 53, the surgical instrument 10 is cleaned and/or disinfected for a first time. For example, the first reprocessing step 53 may involve a manual cleaning. Afterwards, the surgical instrument 10 is transported during a second transport step 54 to another reprocessing area. Then, at a second reprocessing step 55, the surgical instrument 10 is cleaned and/or disinfected for a second time. For example, it is disinfected with the reprocessing apparatus 8. The now disinfected and reprocessed surgical instrument 10 is transported during a third transport step 56 to a storage area. At the storage area, the surgical instrument 10 is dried and stored during a drying and storage step 57 in the storage cabinet 3. When the surgical instrument 10 is required again, it is transported during a fourth transport step 58 back to the surgical area.

The steps of this exemplary reprocessing cycle 50 ensure that the surgical instrument 10 is reprocessed and ready for use in another surgery when required. As the surgical instrument 10 is considered to be disinfected after step 55, any further contamination should be avoided from this point on. However, as the surgical instrument 10 is transported in the transport containers 4 and stored in the storage cabinet 3 during the steps 56, 57 and 58, a recontamination may occur before it is used again in surgery. To prevent this recontamination, at least sections of the inner surfaces 22a, 22b of all the care cycle apparatuses 5, 6 used in the steps 56, 57 and 58 are antimicrobial surfaces 23a, 23b. In other words, after it was reprocessed, the surgical instrument 10 is only accommodated in storage spaces 20a, 20b featuring antimicrobial surfaces 23a, 23b on its inner walls 22a, 22b. A method and a system 1 featuring these care cycle apparatuses 5, 6 efficiently prevents recontamination.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCES

1 system
2 container
3 storage cabinet
4 transport container
5 first care cycle apparatus
6 second care cycle apparatus
7 transport wagon
8 reprocessing apparatus
10 surgical instrument
20a, 20b storage space
21a, 21b wall
22a, 22b inner surface
23a, 23b antimicrobial surface
30 bottom wall
31 side wall
32 top wall
33 door
34 holder
35 drying attachment
40 bottom wall
41 side wall
42 top wall
50 reprocessing cycle
51 surgery step
52 first transport step
53 first reprocessing step
54 second transport step
55 second reprocessing step
56 third transport step
57 drying and storage step
58 fourth transport step

What is claimed is:

1. A method for handling of a surgical instrument in a healthcare facility during a reprocessing cycle, the method comprising:
reprocessing the surgical instrument in at least one reprocessing apparatus for cleaning the surgical instrument,
transporting the cleaned surgical instrument from the at least one reprocessing apparatus to a storage cabinet with a transport container comprising at least one first wall having a first inner surface defining a first storage space for accommodating the surgical instrument, and
storing the transported surgical instrument in a storage cabinet comprising at least one second wall having a second inner surface defining a second storage space for accommodating the surgical instrument,
wherein at least a portion of the first inner surface and second inner surface of the storage cabinet and the transport container is an antimicrobial surface.

2. The method according to claim 1, further comprising, prior to the reprocessing, transporting the surgical instrument to the at least one reprocessing apparatus.

3. The method according to claim 1, further comprising, after the reprocessing, drying the surgical instrument in the storage cabinet.

4. The method according to claim 1, further comprising, after the drying, transporting the surgical instrument with the transport container from the storage cabinet to a surgical area for use in a surgery.

5. The method according to claim 1, wherein
the at least one reprocessing apparatus comprises a first reprocessing apparatus for pre-cleaning the surgical instrument and a second reprocessing apparatus for sterilizing/disinfecting the surgical instrument; and
wherein the reprocessing of the surgical instrument comprises reprocessing the surgical instrument in the second reprocessing apparatus for sterilizing/disinfecting the surgical instrument.

6. The method according to claim 5, further comprising:
prior to the reprocessing of the surgical instrument in the second reprocessing apparatus, transporting the surgical instrument to the first reprocessing apparatus; and
after transporting the surgical instrument to the first reprocessing apparatus, pre-cleaning of the surgical instrument in the first reprocessing apparatus.

7. The method according to claim 6, further comprising:
after pre-cleaning of the surgical instrument in the first reprocessing apparatus;
transporting the pre-cleaned surgical instrument to the second reprocessing apparatus for sterilizing/disinfecting the surgical instrument.

8. The method according to claim 1, further comprising:
treating the first and second surfaces with plasma activation; and
after the treating, coating the plasma treated first and second surfaces with an antimicrobial coating.

9. The method according to claim 1, wherein the coating comprises coating the first and second surfaces with one of a copper or silver coating.
